(19) United States
(12) Patent Application Publication
(10) Pub. No.: US 2021/0244463 A1
(43) Pub. Date: Aug. 12, 2021

(54) ASSEMBLY AID TOOL FOR SECURING ELECTRODES IN A RESECTOSCOPE

(71) Applicant: OLYMPUS WINTER & IBE GMBH, Hamburg (DE)

(72) Inventors: Martin HORN, Hamburg (DE); Hannes MIERSCH, Hamburg (DE); Christian BROCKMANN, Hollenstedt (DE)

(73) Assignee: OLYMPUS WINTER & IBE GMBH, Hamburg (DE)

(21) Appl. No.: 17/251,602

(22) PCT Filed: Jun. 11, 2019
(86) PCT No.: PCT/EP2019/065156

§ 371 Date: Dec. 11, 2020

(30) Foreign Application Priority Data
Jun. 11, 2018 (DE) 10 2018 113 835.0

(51) Int. Cl. A61B 18/14

(52) U.S. Cl. CPC A61B 18/149; A61B 2018/00601; A61B 2018/1407; A61B 18/1482

(57) ABSTRACT

An assembly aid tool for positioning a work instrument in a resectoscope for endoscopic surgery, wherein the assembly aid tool has a grip part in its distal end region, one or more holding elements for retention of a work instrument, and/or bearing faces, in its proximal end region, for supporting a work instrument. In addition, assembly aid systems and resectoscopes include such an assembly aid tool.

### BACKGROUND

The invention relates to an assembly aid tool for positioning a work instrument, preferably an electrode instrument, in a resectoscope, of the type set out in the preamble of claim 1. In addition, the invention also relates to corresponding assembly aid systems and resectoscopes and to methods for using the assembly aid tool.

Resectoscopes are used especially in urology when performing surgical procedures in the bladder and in the urethra. They are usually used for resection and vaporization of tissue, for example tissue in the lower urinary tract. For this purpose, the resectoscopes comprise a longitudinally displaceable work element (e.g. an electrode instrument or a blade instrument) which, after insertion of the resectoscope with its distal work end into the body of the patient, can be pushed forwards out of the distal end of the shaft tube of the resectoscope. At its distal work end, the electrosurgical work element can comprise an electrosurgical electrode in the form of a loop or plasma button. Examples of such instruments are the OES PRO resectoscopes from Olympus.

Customary resectoscopes today have an outer shaft diameter of approximately 26 Charrière (Ch) or more. Despite the already relatively small shaft diameter, the insertion of the shaft portions of such resectoscopes into the urethra causes a not inconsiderable mechanical stress to be exerted on the tissue, sometimes also leading to strictures in the tissue, and efforts are therefore being made to further reduce the diameter of the shafts. In this context, both the diameter of the inner shaft and the diameter of the outer shaft of the resectoscopes have been reduced.

With this reduction of the shaft diameters, it has been found that the customary electrode instruments and other work instruments can no longer be inserted from the proximal direction into the shaft part, and instead they have to be inserted into the inner shaft from the front. During the assembly of the resectoscope, the work instruments therefore have to be grasped in their distal end region by the medical personnel prior to a medical intervention in order to be able to be inserted into the inner shaft and positioned there. The sensitive instruments have to be gripped quite tightly in order to be able to lock the work instrument in the transporter. Since sensitive and easily bendable portions are located in this region, for example the sensitive electrode and easily bendable fork tubes in electrode instruments, incorrect handling can very easily cause bending and damage, as a consequence of which the work instruments are generally rendered unusable. There is therefore a need for an improved assembly method and for instruments that support such a method.

### DESCRIPTION

This object is achieved by an assembly aid tool having the features of claim 1, an assembly aid system having the features of claim 7, a resectoscope having the features of claim 13, and a method, having the features of claim 14, for the positioning of a work instrument. The present invention makes use of an assembly aid tool by which it is possible for a work instrument held thereon to be protected from mechanical stress during insertion into the inner shaft of a resectoscope. At the same time, by using the assembly aid tool according to the invention, there is no need to directly touch the sensitive distal portion of a work instrument, e.g. of an electrode instrument.

In a first aspect, the present invention therefore relates to an assembly aid tool for positioning a work instrument in a resectoscope for endoscopic surgery, characterized in that the assembly aid tool has (a) a grip part in its distal end region, (b) one or more holding elements for retention of a work instrument, and/or (c) bearing faces, in its proximal end region, for supporting the work instrument. Preferably, the assembly aid tool has both the one or more holding elements and also the bearing faces. It is moreover preferable that the one or more holding elements are designed for retention of the fork tubes of a work instrument.

The assembly aid tool makes it easier for the medical personnel to assemble, i.e. put together, the resectoscope prior to a medical intervention. Before the resectoscope is put to use, the assembly aid tool is removed and can be either disposed of or reconditioned for the next use. To make the assembly methods easier, the assembly aid tool can be designed to be used just once (single use) and accordingly disposed of after it has been used. In this way, the use of contaminated or damaged tools can be completely excluded, and the assembly of the instrument can be facilitated to the greatest extent.

The assembly aid tool can be produced from various materials. Since the design as a single-use product means that there are no real requirements in terms of reusability, the assembly aid tool can be produced from plastic for example. It will be appreciated that, when using suitably high-quality plastics, it is also possible for assembly aid tools made of plastic to be designed for repeat use and reconditioning.

The assembly aid tool can be used as part of an assembly aid system which comprises or consists of an assembly aid tool according to the invention and a work instrument, for example an electrode instrument. Accordingly, in a second aspect, the invention relates to an assembly aid system for positioning a work instrument in a resectoscope for endoscopic surgery, characterized in that the assembly aid system comprises a work instrument, preferably an electrode instrument, and an assembly aid tool according to the invention. It is conceivable, for example, to offer work instruments that are pre-fitted in an assembly aid system.

In a third aspect, the invention relates to a resectoscope having an assembly aid tool according to the invention. The assembly aid tools and assembly aid systems according to the invention can be used for positioning a work instrument in such a resectoscope for endoscopic surgery and are accordingly suited for this use. In a customary design, the resectoscope has a tubular shaft. In addition to this shaft part, the resectoscope comprises a grip system for holding and operating it, which grip system usually consists of two grip pieces. The assembly aid tool contained in the resectoscope according to the invention is removed before the resectoscope is put to use.

In its distal end region, the assembly aid tool according to the invention has a grip part. This grip part allows the medical personnel to hold the assembly aid tool firmly with one hand. The grip part can be held between thumb and index finger, for example. No other portions of the assembly aid tool need to be touched during assembly. The work instrument too does not need to be touched during assembly.

Depending on the preferences of the user group, the grip part can be oriented differently with respect to a transverse plane of the assembly aid tool. The transverse plane of the assembly aid tool extends in a transverse plane of a resectoscope when the assembly aid tool is inserted into the inner tube of the resectoscope.

The resectoscope shaft generally comprises an elongate outer tube. An inner tube for receiving an optic and a passage instrument or work instrument is arranged in the inside of the outer tube. According to the invention, the work instrument is preferably an electrode instrument. Alternatively, however, the work instrument can also be an instrument with a blade arrangement at the distal end. Mechanical manipulation of tissue is possible with such instruments. It is moreover conceivable that, in addition to the work instrument, further passage instruments are inserted in the resectoscope.

By virtue of the fact that these work components extending inside the inner tube are separated from the rest of the interior of the outer tube, the components are protected against mechanical stress. It is also conceivable for the inner tube to be designed to be rotatable in the usual way about its longitudinal axis. By rotation of the inner tube, the optic and work instrument at the intervention site can be brought with greater precision to a work position and, if necessary, rotated. The inner tube is therefore preferably mounted rotatably in the outer tube. In addition, the space between outer tube and inner tube can be used so that an irrigation liquid, which has been introduced into the body in order to permit better viewing, can be conveyed back out of the body.

In a spatial orientation, a resectoscope can be divided into different regions by a transverse plane intersecting the shaft tube in the longitudinal direction and horizontally and by a sagittal plane lying perpendicular to the transverse plane, wherein the longitudinal axis of a shaft tube lies both in the sagittal plane and in the transverse plane. The transverse plane intersects the shaft tube transversely and, in a use position of the resectoscope, in a horizontal orientation, and the sagittal plane intersects the shaft tube perpendicularly and, in a use position of the resectoscope, in a vertical orientation. The sagittal plane can in particular lie parallel to a movement plane which is described in a relative movement of grip parts that are mounted pivotably relative to each other on the resectoscope, for example for actuating a carriage of the resectoscope.

In a particularly preferred embodiment, the grip part extends in or parallel to a transverse plane of the resectoscope, i.e. in or parallel to a transverse plane of the assembly aid tool. With this design, the grip part can be used not only to hold the assembly aid tool but also at the same time to ensure that the electrode or blade arrangement arranged below or above, i.e. in a parallel transverse plane, is protected from damage. This is advantageous for all types of usable work instruments, but especially for sensitive and, according to the invention, preferred electrode instruments, particularly those that comprise a loop electrode.

The preferred extent of the grip part in or parallel to a transverse plane does not exclude the possibility of the grip part being able to be adapted ergonomically to being held by human fingers and for this purpose having, for example, a bulge in the upper and/or lower grip face. It is also conceivable that the grip part can be designed sloping slightly from the transverse plane, i.e. at an angle of less than 30°, preferably less than 20°, and/or being curved.

As is indicated, the grip part can have a grip face on mutually opposite sides in order to be held by two fingers, i.e. a lower grip face and an upper grip face in the regular operation of the assembly aid tool.

The assembly aid tool moreover has one or more holding elements for retention of a work instrument, for example for retention of fork tubes of an electrode instrument, and, in its proximal end region, it has bearing faces for supporting the work instrument. While the holding elements serve mainly to stabilize the assembly aid system, the force needed to lock the work instrument in the transporter is transmitted via the bearing faces of the assembly aid tool.

According to the invention, the holding elements can hold the work instrument at any desired location. However, it is preferable that the holding elements hold the work instrument in the region of its shaft part. Retention or holding, as used herein, is therefore to be understood in the sense of securing, particularly in the sense of a releasable securing. The securing can preferably be undone by a simple pulling movement. It is thus preferable that the holding elements can be secured releasably on the shaft part of the work instrument.

The work instruments used can have two fork tubes. Such a set-up is known particularly for electrode instruments. These fork tubes extend quite close to each other in the proximal and the central shaft region of the work instrument, e.g. of the electrode instrument, and it is only in the distal end region of the work instrument that they run apart from each other, such that the distal ends of the fork tubes can receive between them a work element, e.g. an electrode, for example in the form of a loop electrode or a plasma button. The holding elements are preferably designed for retention of fork tubes of a work instrument, i.e. for retention of a work instrument at its fork tubes.

The assembly aid tool preferably extends with its own shaft part from the distal grip part to the proximal bearing faces. The shaft part can have a part-circular cross section. In this way, the assembly aid tool can be inserted for assembly into the inner shaft of a resectoscope, without being impeded by components present in the interior of the inner tube. The one or more holding elements of the assembly aid tool serve for securing the assembly aid tool on the work instrument, in particular for retention and stabilization of the fork tubes.

The holding elements are secured or formed on the shaft part of the assembly aid tool. The holding elements are preferably arranged laterally on the shaft part of the assembly aid tool, in such a way that they are able to contact and hold the shaft part of the work instrument, in particular the fork tubes of a work instrument, in its/their distal end region. For this purpose, the holding elements are at least partly complementary in shape to the shaft part or to one or both of the fork tubes. In preferred embodiments, the assembly aid tool has two holding elements, which are each designed and arranged to hold one of the fork tubes of a work instrument.

The holding elements can be designed as clamping elements, for example. To hold the shaft part, in particular to hold one of the fork tubes, the clamping elements can have a part-circular cross section, wherein the curvature of the inner face of the partial circle corresponds substantially to the curvature of the outer face of the shaft part or of a fork tube. It is conceivable to use clamping elements that have a cross section in the shape of a semicircle or three quarters of a circle, although it has been found that a smaller part-circular cross section is likewise sufficient. For example, the clamping element can have an arc length with a centre angle of 45° to 360°, preferably of 60° to 120°. The part-circular cross section of the clamping elements has in each case at least one opening through which the shaft part or a fork tube is insertable laterally into the clamping element. If the opening is smaller than the diameter of the shaft part or of the fork tube, the clamping element has sufficient flexibility for brief, sufficient enlargement of the opening. In one embodiment, the clamping elements have the function of a snap closure.

Alternatively, the holding elements can completely enclose the shaft part or in each case a fork tube. The shape of the holding element corresponds substantially to that of the clamping elements described above. However, the holding elements do not have an opening through which the shaft part or the fork tubes are inserted laterally. Instead, the connection of these holding elements to the assembly aid tool has in each case a predetermined breaking point. In this way, after successful insertion of the work instrument into the resectoscope, the assembly aid tool can be released from the work instrument by means of the predetermined breaking points being broken. In this embodiment, the holding elements remain on the work instrument after the removal of the assembly aid tool. Such holding elements can be produced, for example, by encapsulation of the shaft part or of the fork tubes by injection moulding.

It is preferable that the assembly aid tool extends substantially parallel to the electrode instrument when both instruments are connected for an assembly aid system. The assembly aid tool thus then extends in a transverse plane which is parallel to a transverse plane of the electrode instrument. The transverse plane in which the distal end region of the fork tubes of the electrode instrument extends is to be designated herein as the fork tube plane.

The clamping elements can hold the shaft part or the fork tubes from different directions, for example from the direction of the transverse plane in which the assembly aid tool extends and/or from the fork tube plane. In the latter case, the clamping elements can be arranged radially next to the fork tubes of a work instrument. In the respective direction, the clamping elements enclose the fork tubes in a part-circular shape. For example, the assembly aid tool can have two clamping elements which grip the fork tubes in each case from the outside or in each case from the inside of the space arranged between the distal end regions of the fork tubes. It is particularly preferable here that the assembly aid tool comprises two clamping elements of part-circular cross section which engage in a part-circular shape around the fork tubes in their distal end region in each case from the inside of the space. It has been found that such an arrangement on the one hand provides a sufficiently firm hold and on the other hand also permits the rapid detachment of the assembly aid tool when the work instrument is locked inside the resectoscope. Depending on the type of work instrument used, however, it may also be advantageous if they are engaged from outside the space. Thus, particularly when using an electrode instrument that has button or needle electrodes, it is advantageous if the assembly aid tool comprises two clamping elements of part-circular cross section which engage in a part-circular shape around the distal end region of the fork tubes, in each case from the outside of the space.

In their distal end region, the fork tubes of conventional electrode instruments have an insulation portion which, for example, can comprise an insulation element made of an electrically insulating material. Suitable materials are known to those skilled in the art and include ceramics, for example. The insulation elements can thicken the fork tubes in the insulation portion.

When the assembly aid system is put together, the clamping elements preferably hold the respective fork tubes of the electrode instrument proximally in relation to the insulation portions, wherein the insulation portions have a greater cross-sectional diameter compared to the average diameter of the respective fork tube in its distal end region outside the insulation portions. Arranging the clamping elements proximally in relation to these insulation portions makes handling of the assembly aid system easier, since the insulation portions prevent the assembly aid tool from slipping in the distal direction.

The pulling force needed to separate assembly aid tool and work instrument is preferably less than the pulling force needed to separate work instrument and resectoscope or transporter. It is thus ensured that, after the work instrument has been latched in the resectoscope or transporter, the assembly aid tool can be released from the work instrument without separating the work instrument again from the resectoscope or transporter.

At the same time, it is preferable that the pulling force needed to separate assembly aid tool and work instrument is greater than the pulling force needed to separate the unlatched work instrument from resectoscope or transporter. This ensures that a separation of assembly aid tool and work instrument, by application of a pulling force, takes place only when the work instrument is fully latched in the resectoscope or transporter. Among other things, the arrangement of the holding elements proximally in relation to insulation portions can contribute to adjusting the desired pulling force.

In its proximal end region, the assembly aid tool has bearing faces for supporting the work instrument. The bearing faces serve to support the work instrument from the distal direction. Accordingly, when the assembly aid system is put together, the bearing faces are arranged distally in relation to that portion of the work instrument that is to be supported. According to the invention, the bearing faces can be designed to support, i.e. contact, the work instrument at its fork tubes, its shaft part, guide plates or other portions. The bearing faces are preferably designed to contact guide plates of the work instrument. Work instruments, in particular electrode instruments, often have one or more guide plates which together form a partial cylindrical shape for receiving the optic. The guide plates are designed and arranged to be complementary in shape to the outer wall of the optic. The elongate optic of a resectoscope runs between the guide plates of the work instrument, e.g. of the electrode instrument.

According to the invention, the assembly aid tool is supported by means of its bearing faces on the distal end of the guide plates or on other parts of the work instrument, such that, by means of the assembly aid tool, a force can be applied in the proximal direction to the work instrument in order to lock the latter in the resectoscope.

The bearing faces are preferably arranged at the proximal end of the assembly aid tool, i.e. the bearing faces form the proximal end of the assembly aid tool. In order to be able to apply a force in the proximal direction to the guide plates or other portions of the work instrument, it is advantageous that the bearing faces extend transversely with respect to the longitudinal axis of the assembly aid tool. Those portions of the work instrument on which the bearing faces are supported also preferably have support faces which extend substantially transversely with respect to the longitudinal axis of the work instrument. The bearing faces can be complementary in shape and size to the distal end of the guide plates or of the support faces of the work instrument. It is also conceivable that the bearing faces protrude radially in one or more directions beyond the distal end of the guide plates or support faces. In this way, even in the event of slight radial displacements, it is possible to ensure that the assembly aid tool safely applies a force and the work instrument is locked. According to the invention, it is at all times ensured that, when the assembly aid system is put together, the bearing faces in the proximal end region of the assembly aid tool bear at least partially on the distal end of one or more guide plates or support faces of the work instrument.

It is conceivable to design the bearing faces on the holding elements that are used according to the invention. In this embodiment, when the assembly aid system is put together, the proximal end of the holding elements could for example be in adjoining contact with a portion of the work instrument (support face or guide plates). The holding elements can in this way be used to apply force to the work instrument from the distal direction.

In a further aspect, the invention relates to a method for positioning a work instrument, preferably an electrode instrument, on a resectoscope, said method comprising steps in which (a) a work instrument is positioned in the shaft part of the resectoscope by means of an assembly aid tool according to the invention, (b) the assembly aid tool is released from the work instrument; and (c) the assembly aid tool is removed from the resectoscope.

The positioning of the work instrument comprises the insertion in a correct orientation and preferably also the locking of the work instrument in the resectoscope, preferably in the transporter of the resectoscope.

The assembly aid tool can be released from the work instrument by means of a slight movement of said assembly aid tool transversely or substantially transversely with respect to the fork tube plane. By virtue of the preferred part-circular design of the holding elements or by virtue of the holding elements being designed with predetermined breaking points, no great force needs to be applied in order to do this. The locking of the work instrument is therefore not adversely affected by the release of the assembly aid tool. As has been described above, a further factor preventing the locking of the work instrument from being adversely affected is preferably that the pulling force for separating the assembly aid tool and the work instrument is less than the pulling force for separating the work instrument, locked in the resectoscope or transporter, and the resectoscope or transporter.

After the assembly aid tool has been released from the work instrument, the assembly aid tool can be easily removed from the shaft of the resectoscope and either disposed of or reconditioned.

### BRIEF DESCRIPTION OF THE FIGURES

Illustrative embodiments of the invention are presented schematically in the drawings, in which:

FIG. 1 shows a schematic side view of a resectoscope;

FIG. 2 shows a schematic side view of an assembly aid system according to the invention;

FIG. 3 shows a schematic perspective view of a portion of an assembly aid system according to the invention;

FIG. 4 shows a schematic perspective view of a portion of an assembly aid system according to the invention inserted into the inner tube of a resectoscope;

FIG. 5 shows a further schematic perspective view of a portion of the assembly aid system according to the invention from FIG. 3; and

FIG. 6 shows a further schematic perspective view of a portion of the assembly aid system according to the invention from FIG. 4, with the assembly aid system inserted into the inner tube of a resectoscope.

### ILLUSTRATIVE EMBODIMENTS

Further advantages, characteristics and features of the present invention will become clear from the following detailed description of illustrative embodiments in which reference is made to the accompanying drawings. However, the invention is not limited to these illustrative embodiments.

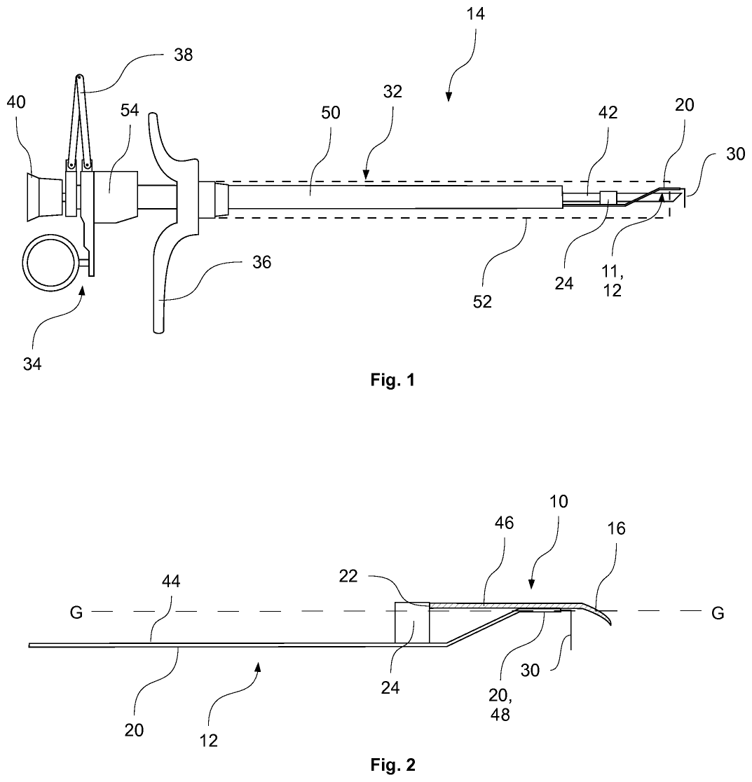

FIG. 1 shows a highly schematic side view of a resectoscope 14 from the prior art. The assembly aid tool 10 according to the invention can be used to insert a work instrument 11 into the resectoscope 14. The work instrument 11 is designed here as an electrode instrument 12. The insertion of the assembly aid system 26 takes place in particular from the distal end of the shaft part 32 of the resectoscope 14. In the resectoscope 14 depicted, the electrode instrument 12 has already been inserted into the resectoscope 14 and locked there. It will be seen that the electrode instrument 12 has a loop electrode 30, which is held by fork tubes 20. The fork tubes 20 are connected to the optic 42 by means of guide plates 24. By means of the loop electrode 30, tissue can be removed by electrosurgical ablation. Here, a high-frequency electrical voltage is applied to the loop electrode 30 in order to cut tissue.

Within the shaft region 32 of the resectoscope, the optic 42 and the electrode instrument 12 extend inside an inner tube 50 which, for example, can be mounted rotatably with respect to the outer tube 52. An embodiment is shown in which the electrode instrument 12 and the optic 42 protrude slightly beyond the distal end of the outer tube 52. However, a great many variations are conceivable in this regard. The inner tube 50, as shown, can also be slightly shorter than the outer tube 52, or it can also be of the same length or longer. A space between the inner tube 50 and the outer tube 52 can serve as a return conduit for irrigation liquids and/or body fluids.

An eyepiece or alternatively a camera for viewing the treatment region can be arranged on the proximal end of the optic 42. At the distal end of the optic 42, the latter has, for example, a lens (not shown) which is targeted on the region in which surgery is to be performed. However, the optic 42 can also be assigned an electronic sensor, for example a CCD chip.

The resectoscope 14 shown has a passive transporter in which, by relative movement of the grip pieces 34 and 36 arranged proximally from the shaft part 32, the carriage 54 is displaced in the distal direction towards the distal, first grip piece 34 counter to a spring force applied by a spring bridge 38. In the displacement of the carriage 54 in the distal direction towards the grip piece 34, the electrode instrument 12 and/or the optic 42 is forced in the distal direction in a manner not shown. Upon relaxation of the grip pieces 34, 36, the spring force generated by the spring bridge 38 forces the carriage 54 back to its rest position, wherein the shaft part 32, and thus also the electrode instrument 12 and/or the optic 42, is pulled in the proximal direction. Upon the rearward displacement of the carriage 54, an electrosurgical intervention can be performed with the electrode instrument 12 without manual force from the operator, i.e. passively.

FIG. 2 shows a schematic side view of an assembly aid system 26 according to the invention, in which an assembly aid tool 10 (indicated by hatching) is connected to an electrode instrument 12. The connection between the assembly aid tool 10 and the electrode instrument 12 is effected via clamping elements 18 (not shown in this view) and the bearing faces 22 adjacent to the distal end of the guide plates 24.

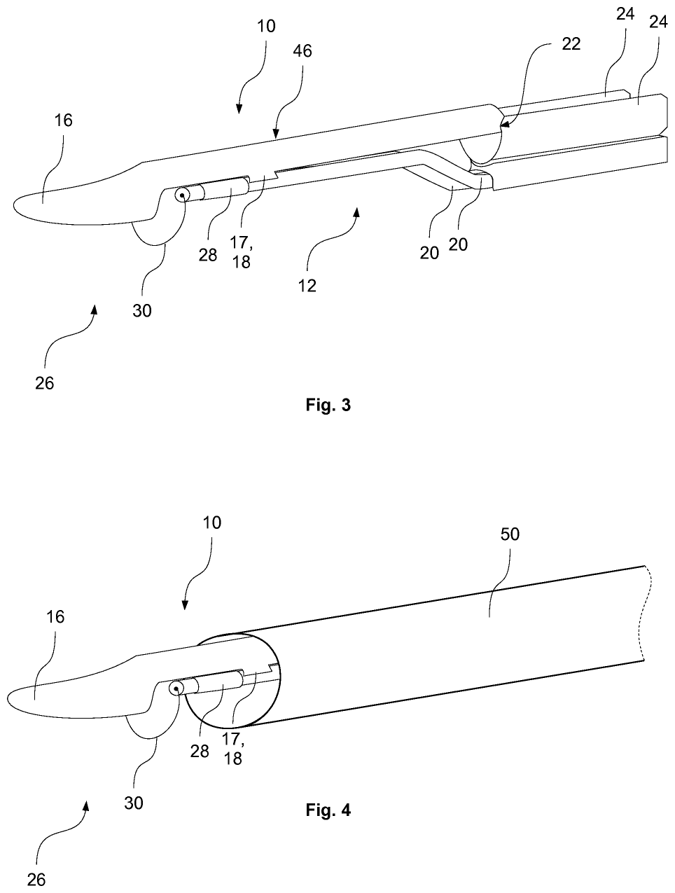

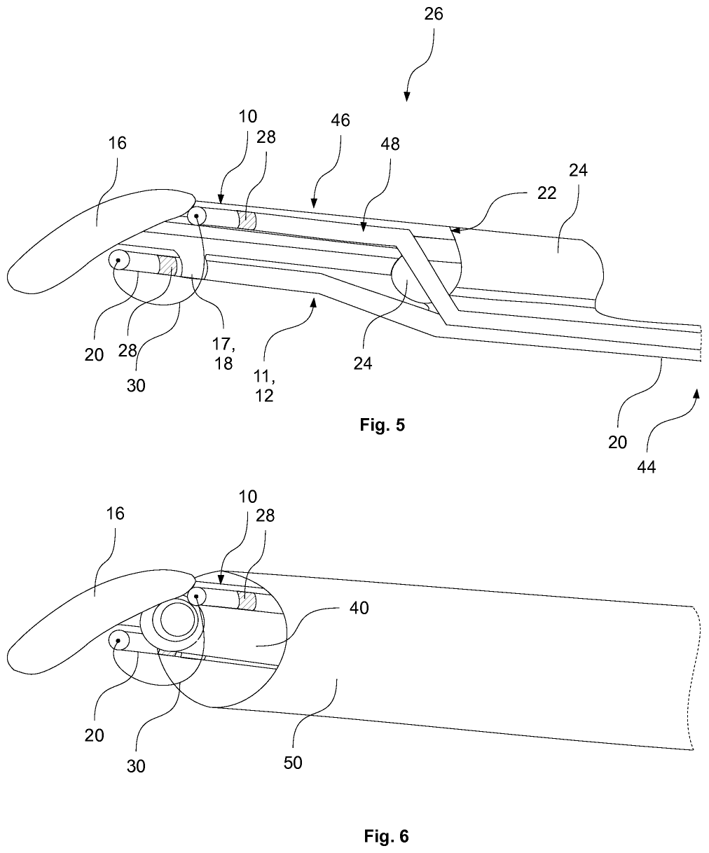

As can also be seen in FIGS. 3 and 5, the assembly aid tool 10 has an elongate configuration with a shaft part 46 and has, at its distal end, a grip part 16 shaped anatomically for holding between two fingers. The shaft part 46 has a part-circular cross section, with which it is adapted optimally to the curves in the inner tube 50. The electrode instrument 12 has fork tubes 20 which, in their distal end region, are angled into a fork tube plane G.

FIGS. 3 to 6 each show the one portion of the assembly aid system 26 according to the invention, wherein the system in FIGS. 4 and 6 is inserted into the inner tube 50 of a resectoscope 14. To simplify matters, the outer tube 54 of the resectoscope 14 is not shown in these views. FIGS. 3 and 4 show an embodiment of the assembly aid system 26 according to the invention from a different perspective than in FIGS. 5 and 6.

Like the assembly aid system 26 shown in FIG. 2, the embodiment in FIGS. 3 to 6 has an assembly aid tool 10 which is connected to an electrode instrument 12. In its distal end region, the assembly aid tool 10 comprises an ergonomically shaped grip part 16 via which the assembly aid system 26 can be grasped. By means of this grip part 16, the assembly aid system 26 can be held securely between two or more fingers. The system does not need to be touched at any other points. The danger of damaging the electrode instrument 12 is greatly reduced in this way.

The assembly aid tool 10 moreover has an elongate shaft part 46 on which two holding elements 17 are arranged laterally, only one of these holding elements 17 being visible in each of the views shown. The holding elements 17 are designed as clamping elements 18. The clamping elements 18 are partly complementary in shape to the fork tubes 20 and are able to hold these fork tubes 20 with a clamping action. Alternatively, however, it is also conceivable to design the holding elements 17 such that they hold the work instrument 11 at another position of its shaft part.

At the proximal end of the assembly aid tool 10, two bearing faces 22 are formed in the direction of the distal end of the electrode instrument 12. The bearing faces 22 are partly complementary in shape to the distal end of the guide plates 24 of the electrode instrument 12. In this way, a force sufficient for locking purposes can be exerted on the guide plates 24 by means of the assembly aid tool 10 from a distal direction.

The guide plates 24 together form a structure having, in the longitudinal direction of the electrode instrument, a part-circular cross section. As can be seen from FIG. 6, the optic 42 can be received in this structure. The assembly aid system 26 is designed such that the optic 42 can be guided through between the assembly aid tool 10 and the guide plates 24 and also between the two fork tubes 20.

The electrode instrument 12 moreover has two fork tubes 20, which extend substantially over the entire length of the electrode instrument 12. In the proximal and the central region of the shaft part 32, the fork tubes 20 lie close to each other and, in the distal region of the shaft part 32, they are then guided away from each other and to the opposite side of the inner shaft 50 in the manner shown in FIGS. 3 to 6. As a result, the electrode at the distal end of the electrode instrument 12, which electrode is here designed as a loop electrode 30 and has been inserted into an inner tube 50, is arranged in a space-saving manner within the inner tube 50 in front of the optic 42. Before and during the insertion and positioning of the assembly aid system 26, the electrode is moreover protected from damage by virtue of the grip part 16 lying over it, i.e. in a parallel transverse plane.

The clamping elements 18 are positioned on the shaft part 46 of the assembly aid tool 10 in such a way that, when an assembly aid system 26 is put together, they lie proximally in relation to the insulation portions 28 which are usually arranged at the distal end region of fork tubes 20. In this way, slipping of the assembly aid tool 10 from the electrode instrument 12 during insertion is prevented.

Although the present invention has been described in detail on the basis of the illustrative embodiments, it is obvious to a person skilled in the art that the invention is not restricted to these illustrative embodiments but rather that modifications are possible, whereby individual features can be omitted or different types of combinations of the individual features shown can be implemented, without departing from the scope of protection of the accompanying claims. The present disclosure covers all combinations of the individual features shown.

### LIST OF REFERENCE SIGNS

10 assembly aid tool
11 work instrument
12 electrode instrument
14 resectoscope
16 grip part
17 holding element
18 clamping element
20 fork tube
22 bearing face
24 guide plate
26 assembly aid system
28 insulation portion
30 loop electrode
32 shaft part of resectoscope
34 first grip piece
36 second grip piece
38 spring bridge
40 eyepiece
42 optic
44 shaft element of electrode instrument
46 shaft part of assembly aid tool
48 distal end region
50 inner tube
52 outer tube
54 carriage
G fork tube plane

1. An assembly aid tool for positioning a work instrument in a resectoscope for endoscopic surgery, wherein the assembly aid tool has
(a) a grip part in its distal end region,
(b) one or more holding elements for retention of a work instrument and/or
(c) bearing faces, in its proximal end region, for supporting a work instrument.

2. The assembly aid tool according to claim 1, wherein the assembly aid tool is suitable for the positioning of an electrode instrument.

3. The assembly aid tool according to claim 1, wherein the bearing faces are designed for the contacting of guide plates of an electrode instrument.

4. The assembly aid tool according to claim 1, wherein the holding elements are designed as clamping elements.

5. The assembly aid tool according to claim 1, wherein the assembly aid tool is made from a plastic.

6. The assembly aid tool according to claim 1, wherein the bearing faces form the proximal end of the assembly aid tool.

7. An assembly aid system for positioning a work instrument in a resectoscope for endoscopic surgery, wherein the assembly aid system comprises a work instrument and an assembly aid tool according to claim 1.

8. The assembly aid system according to claim 7, wherein the work instrument is an electrode instrument.

9. The assembly aid system according to claim 8, wherein the electrode instrument comprises two fork tubes, which are held by the one or more holding elements of the assembly aid tool.

10. The assembly aid system according to claim 9, wherein the fork tubes have an insulation portion in their distal end region, and the holding elements of the assembly aid tool hold the respective fork tubes proximally in relation to the insulation portions.

11. The assembly aid system according to claim 8, wherein the bearing faces in the proximal end region of the assembly aid tool bear at least partially on the distal end of one or more guide plates of the electrode instrument.

12. The assembly aid system according to claim 8, wherein the electrode instrument comprises a loop electrode.

13. A resectoscope with assembly aid tool according to claim 1.

14. A method for positioning a work instrument on a resectoscope, the method comprising steps in which
(a) a work instrument is positioned in the shaft part of the resectoscope by means of an assembly aid tool according to claim 1;
(b) the assembly aid tool is released from the work instrument; and
(c) the assembly aid tool is removed from the resectoscope.
